(19) United States
(12) Patent Application Publication
(10) Pub. No.: US 2011/0091588 A1
(43) Pub. Date: Apr. 21, 2011

(54) ORCHID VANDA COERULEA AS A COSMETIC ACTIVE AGENT

(75) Inventors: Jean-Hubert CAUCHARD, Orléans (FR); Françoise Pellicier, Loury (FR); Jean-Christophe Archambault, Meung Sur Loire (FR); Frédéric Bonte, Orléans (FR)

(73) Assignee: LVMH RECHERCHE, Saint Jean de Braye (FR)

(21) Appl. No.: 12/707,337

(22) Filed: Feb. 17, 2010

(30) Foreign Application Priority Data
Oct. 16, 2009 (FR) 0957289

(51) Int. Cl. A61K 8/97; A61K 8/02; A61Q 19/08; A61Q 19/00

(52) U.S. Cl. 424/773; 424/725; 424/774; 424/779; 424/401

(57) ABSTRACT

An extract of the orchid Vanda coerulea is used as cosmetic active agent for regulating the cell cycle in the skin to combat or delay the appearance of visible signs of skin aging or slow down the effects thereof. The extract is used in a cosmetic composition or in an anti-aging method for delaying the appearance of signs of skin aging or slowing down the effects thereof to maintain the firmness of the skin and tissues or restore a more radiant complexion to the skin or alternatively combat disorders of pigmentation related to skin aging.

### CROSS-REFERENCE TO RELATED APPLICATION(S)

This application claims benefit of Ser. No. 0957289, filed Oct. 16, 2009 in France and which application(s) are incorporated herein by reference. A claim of priority to all, to the extent appropriate is made.

### BACKGROUND OF THE INVENTION

1. Field of the Invention

The invention relates to an extract of the orchid Vanda coerulea as cosmetic active agent for regulating the cell cycle in the skin, in particular for alleviating or delaying the appearance of signs of skin aging or slowing down the effects thereof.

2. Prior Art

Skin aging is expressed in numerous intrinsic changes reflected by the gradual appearance of visible signs which modify the visible quality and beauty of the skin. It is known that various ways exist today of combating skin aging and that it is difficult to act on several factors simultaneously. However, it is this which a person skilled in the art wishes to do. Factors such as the modification of the differentiation of the epidermis, the slowing down in cell proliferation, the loss of skin elasticity, the less dense dermis, the decrease in hydration or disorders of pigmentation result with age in skin which is finer, drier, less supple, rougher and less radiant (Rocquet et al., Acta Dermato. venereol., Vol. 2002, 11 (3), 71-93). The skin grain is coarser and numerous skin imperfections grow and become visible. It is therefore particularly advantageous to combat cell aging long before its process.

The entry into senescence of skin cells is related in particular to detrimental changes and imbalances in biochemical or morphological events occurring during cell proliferation.

The cell cycle, which represents the combination of these events, is divided mainly into four phases represented diagrammatically according to FIG. 1.

These phases respectively reflect:
the preparation of the cells for DNA replication (G1 phase, or first phase of cell growth): the cell DNA content is identical to the content of the cells at rest. This G1 phase, which depends on the cell type, is often responsible for the variability in the interphase and thus in the cell cycle,
DNA replication (S phase),
the preparation of the cells for mitosis (G2 phase or second phase of cell growth): this phase is characterized by an increase in RNA and proteins. The cell has duplicated its initial genetic material and is preparing for division by initiation of chromatin modification.
cell division or mitosis (M phase), on conclusion of which the daughter cells have their own RNA and protein contents.

The cells at rest, in a quiescent stage of non-division, are in a “G0” phase, which corresponds to departure from the cell cycle and generally occurs during the G1 phase.

A person skilled in the art understands that these major stages obviously do not correspond to processes with perfectly defined boundaries: some protein syntheses take place throughout the cell cycle.

“Senescent” cells are blocked in G0 or G1 phase of the cell cycle and do not divide further. They become incapable of reentering S phase and no longer respond to stimulation by physiological mitogenic agents (Jenkis, Mech. Age Dvpt., 2002, 123, 801-10).

This fall in cell activity can be explained by the selective repression of genes involved in the progression of the G1 phase and in the synthesis of DNA or, conversely, by the overexpression of inhibitors of cyclins, proteins which regulate the cell cycle.

This accumulation of senescent cells results on the whole in a gradual decline in the functions and integrity of the skin tissue (Camas' J., 1996, Cell, 84, 497-500). Senescent fibroblasts, for example, secrete more metalloproteinases and produce less collagen (Jenkis G., Mech Age Dvpt., 2002, 123, 801-10), which results in the detrimental change in the extracellular matrix.

### SUMMARY OF THE INVENTION

The main aim of the invention is to solve the new technical problem consisting in providing a cosmetic active agent intended to combat, or slow down, the effects of skin cell aging, in particular by acting upstream from this process.

Another aim of the invention is to solve the new technical problem consisting in providing a cosmetic active agent intended to regulate the skin cell cycle, in particular in order to combat, or delay, the appearance of signs of skin aging or to slow down the effects thereof.

A further aim of the present invention is to provide an anti-aging cosmetic active agent aiming at maintaining the firmness of the skin and tissues, at rendering the complexion of the skin more radiant or at combating disorders of pigmentation related to aging of the skin.

Another aim of the present invention is to provide an anti-aging cosmetic composition and an anti-aging cosmetic care method.

A final aim of the invention is to solve all the above technical problems by a solution which is particularly simple, which is relatively inexpensive and which can be used on an industrial scale.

### SUMMARY OF THE INVENTION

According to a first aspect, the invention relates to the use, in a cosmetic composition, of an extract of the orchid Vanda coerulea intended to regulate the skin cell cycle and in particular to increase the percentage of skin cells in the phase of replication of the genetic information or to reduce the percentage of senescent cells among the total population of skin cells or to improve the biological activity of the cells of the skin tissue detrimentally affected by aging or as cosmetic active agent intended to delay the appearance of signs of skin aging or to slow down the effects thereof and in particular to maintain the firmness of the skin and tissues or to render a skin complexion more radiant or else intended to combat disorders of pigmentation related to aging of the skin.

According to a second aspect, the invention relates to an anti-aging cosmetic care method aiming in particular at maintaining the firmness of the skin and tissues or rendering a skin complexion more radiant or for combating disorders of pigmentation relating to skin aging, which comprises the application, to the part of the skin of the face or body concerned, of an effective amount of a cosmetic composition comprising an extract of the orchid Vanda coerulea.

### BRIEF DESCRIPTION OF THE DRAWINGS

FIG. 1 diagrammatically represents the cell cycle.

FIG. 2 represents the distribution of skin cells in each of the various phases of the cell cycle which is obtained for the various tests (controls and treatments) carried out on “young” NHFs (F3P) or “aged” NHFs (F22P).

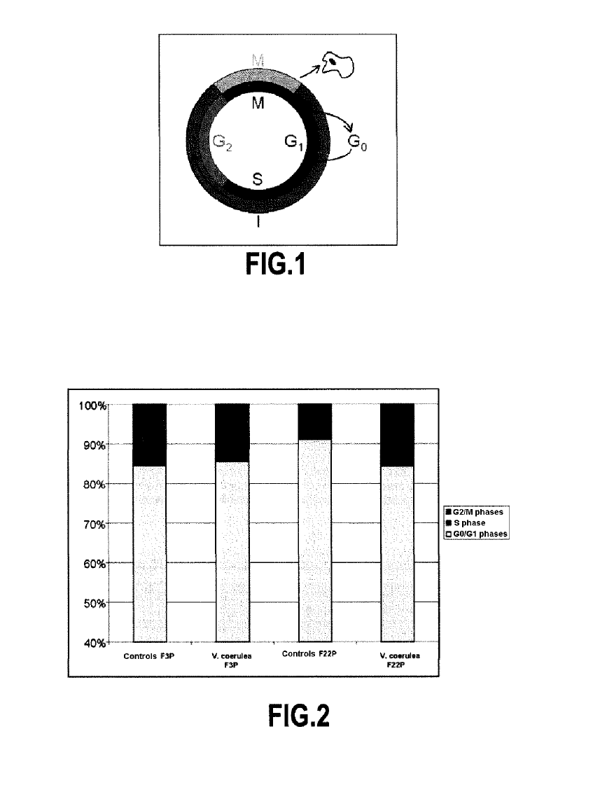

### DESCRIPTION OF THE INVENTION

The inventors have demonstrated in vitro that, in a population of “young” skin cells which have divided only a small number of times, the percentage of active cells, that is to say in the phase of replication of the genetic information (S phase), is significantly higher in proportion than in a population of “aged” cells which have divided a larger number of times.

This demonstrates the negative effect of cell aging on the ability of skin cells to divide and proliferate for the purpose of maintaining the mechanical properties of the skin and its role of effective barrier with regard to environmental stresses.

Reducing the relative proportion, among the total population of cells, of senescent cells to the advantage of cells which actively divide has the major consequence of restoring the biological mechanisms and functions of aged cells to a level similar to that of young cells.

A cosmetic active agent capable of restoring, to cells, this ability to actively divide, which makes it possible to long before combat the effects of skin aging, while providing for the maintenance of the biochemical potential of the cell machinery, would be an anti-aging active agent of particular importance, with an effect on all the biological functions of the cells on which it acts.

It has now been discovered, completely unexpectedly, that extracts of the orchid Vanda coerulea exhibit such properties.

An advantageous distinguishing feature of the extract of the invention is that of limiting the phenomenon of cell senescence and of stimulating the mechanisms which make it possible to maintain a large number of nonsenescent active skin cells.

Patent application FR 0851 382 describes the use, in a cosmetic composition, of an extract of at least a part of an orchid of the species Vanda coerulea as agent intended to maintain or restore the state of hydration of the skin. However, no mention is made of an anti-aging activity.

Furthermore, an extract of the whole Vanda coerulea plant, used as antioxidant in cosmetic compositions, is known.

However, here again, the active principle is not described for its anti-aging effect with a cell action upstream from the aging process.

The inventors have now discovered the properties of an extract of the orchid Vanda coerulea for regulating the processes of senescence and its consequences, in particular with regard to aged normal human fibroblasts (NHF).

As explained above, the treatment of “aged” fibroblasts with a Vanda coerulea extract makes it possible to increase the percentage of cells in the phase of active replication of the genetic information (S phase) to a level equivalent to that of “young” fibroblasts of the dermis.

The extract thus acts as regulator of the cell cycle, with the consequence of having more cells in the active phase of synthesis having the ability to multiply in the skin tissue to give daughter cells.

This important and surprising effect of the Vanda coerulea extract makes it possible to increase the biological activity potential of all the dermal fibroblasts in order to effectively combat a decline in the functions and integrity of skin tissue and to protect the matrix environment in the tissues, which is fundamental for their quality.

A first subject matter of the present invention relates to the use, in a cosmetic composition, of an extract of the orchid Vanda coerulea as active agent intended to delay the appearance of signs of skin aging or to slow down the effects thereof, in particular to maintain the firmness of the skin and tissues or to render a skin complexion more radiant, or else intended to combat disorders of pigmentation related to aging of the skin.

The invention relates, according to another aspect, to the use, in a cosmetic composition, of an extract of the orchid Vanda coerulea as active agent intended to regulate the cycle of the skin cells, in particular to increase the percentage of skin cells in the phase of replication of the genetic information and to reduce, at the same time, the percentage of senescent cells among the total population of skin cells, or else to improve the biological activity of skin cells detrimentally affected by aging.

The plant material used can be the whole plant or a part of the plant, such as the leaves, stem, flowers or roots.

The extract is preferably obtained from the stems and/or roots and/or leaves of the orchid, preferably the stems.

The extract is prepared by various extraction processes known to a person skilled in the art.

However, extraction is preferably carried out by bringing the selected plant material into contact with a polar solvent or a mixture of polar solvents.

Prior to the stage of extraction itself, the plant material may have been dried and/or ground.

According to a preferred implementation of the extraction, the plant material occurs in the dry and ground state.

The choice is advantageously made, as polar solvent or mixture of polar solvents, of a solvent or a mixture of solvents chosen from water, a C₁-C₄ alcohol, for example ethanol, a C₂ to C₆ glycol preferably chosen from glycerol, butylene glycol and propylene glycol, and any one of their mixtures.

According to a preferred implementation of the invention, extraction is carried out using an aqueous/alcoholic mixture, preferably a mixture comprising 90% v/v of water and 10% v/v of ethanol.

According to another alternative form of the invention, extraction can be carried out by a process employing a polar solvent in the subcritical state, said solvent advantageously being water.

Extraction can also optionally comprise at least one additional stage of decoloration or purification of the extract which can comprise, for example, a treatment of the extract with a solution of a polar solvent or a mixture of polar solvents, for example an ethanol/water (70/30 v/v) solution, in the presence of particles of active charcoal.

The decoloration can also comprise a treatment of the extract with a nonpolar solvent, for example a C₆-C₇ alkane, or else with CO₂ in the supercritical state.

Extraction can be completed by a stage of partial or complete removal of the extraction solvents.

In the first case, the extract is generally concentrated until an aqueous concentrate devoid of a significant amount of organic solvents is obtained; in the second case, a dry residue is obtained.

Alternatively, the product from the extraction stage can be lyophilized or atomized in order to be provided in the form of a powder.

Use is made, as active agent in the cosmetic composition according to the invention, either of the powder in this state or of a solution or a suspension of said powder in a cosmetically acceptable solvent or a mixture of cosmetically acceptable solvents which can be identical to or different from that used for the extraction.

The cosmetic composition comprises an effective amount of extract to obtain the desired effect.

The composition thus preferably comprises from 0.001% to 5% by dry weight of extract, preferably from 0.1% to 2% by weight.

The tests carried out by the inventors have shown that the properties of the extract can also be obtained or improved in cosmetic compositions in which the extract of the orchid Vanda coerulea is combined with other active agents in the form of purified molecules and/or plant extracts exhibiting similar and/or complementary cosmetic effects to that of said extract.

The composition can thus comprise one or more other plant extracts obtained from whole plants or from plant parts.

The cosmetic composition can thus advantageously comprise at least one extract of another orchid, in particular an extract of another species of orchid belonging to the Vanda genus, such as the orchid Vanda teres, or an extract of an orchid belonging to the genus Brassocattleya, such as the orchid Brassocattleya marcella Kos, or else an extract of an orchid belonging to the genus Phalaenopsis, such as the orchid Phalaenopsis amabilis.

The cosmetic composition can in addition also comprise one or more other cosmetic active agents chosen from the group of substances having an anti-aging activity; of substances having a depigmenting activity or a lightening activity on the skin; of substances having a slimming activity; of substances having a hydrating activity; of substances having a calming, soothing or relaxing activity; of substances having an activity in stimulating skin microcirculation in order to improve the radiance of the complexion, in particular of the face; of substances having a seboregulating activity, for the care of greasy skin; of substances intended to clean or purify the skin; or of substances having an activity in combating free radicals.

In particular, the cosmetic composition can comprise one or more other cosmetic active agents chosen from:
agents which promote cell replacement, such as vitamin A (retinol) and/or its esters, in particular a vitamin A propionate; α- or β-hydroxy acids, such as fruit acids, malic, glycolic or citric acid, salicylic acid or its esters, or gentisic acid or its esters, in particular tocopherol gentisate;
agents which stimulate the firmness of the skin, such as peptides which stimulate the synthesis of collagen, in particular type I, II, IV or VII collagen, a Centella asiatica extract, madecassic acid, asiatic acid, madecassoside, an oats extract, a Bertholletia exscelsa extract, a protein hydrolysate, soy peptides, a Potentilla erecta extract, a Siegesbeckia orientalis extract, or ginsenosides or notoginsenosides in particular Rb1, R0;
agents which regulate the differentiation of the epidermis, including those of the following types: ecdysteroids, ecdysterone, turkesterone or calcium derivatives or vitamin D precursors;
adenosine, or carnitine or its derivatives;
metalloproteinase inhibitors, in particular MMP1, MMP2 or MMP9 inhibitors, such as a Ruscus aculeatus extract or flavonoids, such as quercetin, kaempferol, apigenin or wogonin, or plant extracts comprising them;
elastase inhibitors, such as Aspergillus fumigatus, Momordica charantia or Cucurbita maxima extracts;
agents which stimulate the synthesis of dermatopontin, such as an amber extract;
agents which close the pores, such as extracts of astringent plants, for example a Hamamelis extract;
screening agents which protect from UV-A and UV-B radiation, such as benzophenone, 4-butyl nnethoxydibenzoylmethane, octocrylene, ethylhexyl methoxycinnamate, ethylhexyl salicylate, phenylbenzinnidazole sulfonic acid or homosalate, alone or in combination with titanium oxides;
agents intended to combat disorders of pigmentation, in particular those related to skin aging, such as kojic acid, blackberry or licorice root extracts, arbutin, calcium pantothein sulfonate, boldine, diacetylboldine, vitamin C and its derivatives, in particular glycosides or lily extracts, in particular lily bulb extracts;
agents for combating free radicals or anti-inflammatory agents, such as an Artemisia capillaris extract, a Sanguisorba officinalis extract, resveratrol and its derivatives, turmeric or curcumin or tetrahydrocurcumin, polyphenols extracted from grape seeds, vitamin E and its derivatives, in particular its phosphate derivatives, ergothioneine or its derivatives, or idebenone;
agents which promote the synthesis of hyaluronic acid in the epidermis and dermis or of glycosaminoglycans, to give hydrated and plumped skin, in particular an Eriobotrya japonica extract or hyaluronic acid fragments of low molecular weight or also an Adenium obesum extract;
magnesium aspartate, for improving the antiwrinkle action;
D-xylose, for improving the plumping effect for plumped skin;
and any one of their mixtures.

Advantageously, the composition additionally comprises at least one cosmetically acceptable excipient which can be chosen from pigments, dyes, polymers, surface-active agents, rheology agents, fragrances, electrolytes, pH adjusters, antioxidants, preservatives and their mixtures.

The cosmetic composition can be, for example, a serum, a lotion, a cream or else a hydrogel, preferably a mask, or also provided in the form of a stick or of a patch.

The extract and the composition of the invention exhibit an effect, which is particularly desired, of delaying the appearance of signs of skin aging or slowing down the effects thereof; in particular of maintaining the firmness of the skin and tissues or rendering a skin complexion more radiant or else of combating disorders of pigmentation relating to skin aging.

Another subject matter of the invention is an anti-aging cosmetic care method, which comprises the application, to at least a part of the skin of the face or body, of an effective amount of a cosmetic composition comprising an extractive the orchid Vanda coerulea in order to delay the appearance of signs of skin aging or to slow down the effects thereof; in particular in order to maintain the firmness of the skin and tissues or to restore a more radiant complexion to the skin or alternatively to combat disorders of pigmentation related to skin aging.

Advantageously, the cosmetic composition is applied to an area of skin of the body or face exhibiting visible signs of aging, such as the presence of wrinkles or fine lines, a loss of skin elasticity, a reduction in the thickness of the skin, an increase in skin dryness or roughness, a loss of skin suppleness or radiance, or alternatively visible signs of a disorder of pigmentation related to skin aging, such as a senile lentigo.

In the examples, all the percentages are given by weight, the temperature is in degrees Celsius and the pressure is atmospheric pressure, unless otherwise indicated.

Other aims, characteristics and advantages of the invention will become clearly apparent in the light of the explanatory description which will follow, made with reference to examples of the preparation of extracts and of tests demonstrating the properties of the extracts and to examples of cosmetic compositions using such an extract, which are given by way of illustration of the invention without, however, limiting the scope thereof.

### EXAMPLES

### Example 1

### Preparation of an Extract of Vanda Coerulea Stems

a) Preparation of an Extract of Stems

The extract is prepared from Vanda coerulea stems in the dry and ground state.

10 g of the plant are introduced into a 250 ml round-bottomed flask, to which 150 ml of an ethanol/water (90/10 v/v) mixture are added.

The round-bottomed flask, surmounted by a bulb condenser and under magnetic stirring, is heated in a thermostatically-controlled bath, up to reflux of the solvent, and is kept stirred for 30 minutes.

The round-bottomed flask is subsequently allowed to cool to ambient temperature.

The mixture is subsequently filtered under vacuum on a Buchner funnel with a GF/F Whatman 70 μm filter and a tared flask. The cake is washed on the Buchner funnel with 50 ml of the extraction solvent.

The filtrates are combined, weighed, introduced into a pretared round-bottomed flask and then concentrated to dryness on a rotary evaporator in a water bath placed at a maximum temperature of 50° C.

b) Preparation of an Extract of Roots

The same protocol is used to prepare an extract of Vanda coerulea roots (extract No. 2).

These two extracts will be used in the examples which follow, either separately or as a mixture and combined in different proportions.

### Example 2

### Measurement of the Phases of the Cell Cycle on Human Dermal Skin Cells by Flow Cytometry (FCM)

The fibroblasts are obtained from biopsies of healthy skin of consenting patients. Their in vitro aging is obtained by successive subculturing as described by Hayflick (Exp Cell Res 1965, 37:614-636). The “Hayflick” model is a model of in vitro cell aging. These cells, aged in vitro by successive passages in the presence of trypsin, exhibit identical characteristics to those of cells resulting from elderly patients.

The fibroblasts which have been subjected to only three passages in the presence of trypsin, denoted F3P, have an ability to divide equivalent to that of young cells, whereas fibroblasts which have been subjected to a greater number of passages, in the present case 22 passages, exhibit an ability to proliferate equivalent to that of aged cells. In the latter case, the fibroblasts are denoted F22P.

Equipment and Methods

FCM offers a methodology for the analysis of the cell cycle. It makes it possible to study the distribution of cells in the various phases of the cell cycle.

The isolated and suspended cells are entrained in a liquid stream. The aligned cells pass at high speed in front of a laser. Light scattering, either in the axis of the laser or in 90°, gives information on the size and structure of each cell. The cytometric analysis is preceded by a stage of labeling with fluorescent molecules that are specific for a cell structure or function. These molecules emit a measurable fluorescence after excitation by the incident light source. The scattering and fluorescence signals are captured by detectors, which convert them into electrical signals processed by a computer system. Each cell thus represents an “electrical event” with several coordinates (size, structure, fluorescence 1, fluorescence 2, and the like).

It is possible to determine the percentage of cells in the different phases of the cell cycle using labeling with propidium iodide, which is an intercalating agent both for DNA double strands and for RNA double strands. Hydrolysis of the RNA by ribonuclease is necessary before the staining of the DNA by the intercalating agent.

Cell Culturing

A large amount of cells (approximately 1 million cells/ml for each analytical condition) is necessary for an FCM analysis. The cells are detached from the support using trypsin, individualized and then labeled before being analyzed.

Culturing of the NHFs

The primary cultures of normal human fibroblasts (NHFs) are prepared from dermis explants.

The NHFs are cultured for one week in 75 cm³ flasks in the presence of MEM (Modified Eagle's Medium)+10% FCS (Fetal Calf Serum); the medium is changed every two days. The cells are treated at confluence.

Treatment of the Cells

The cells are treated for 24 hours with the active agent of the test, in the present case an extract of stems of the orchid Vanda coerulea, the stock solutions of which are prepared at 50 mg/ml in DMSO and used at 25 μg/ml after dilution.

Staining the DNA with Propidium Iodide

A 0.25% trypsin-EDTA solution is used to detach the cells. The flasks are rinsed with 5 ml of PBS without Ca²⁺ or Mg²⁺ and then 2 ml per flask of trypsin-EDTA are added, distributed over the whole of the cell layer.

The cells are incubated at 37° C., 5% CO₂.

When the cells are floating, 10 ml of MEM medium+10% FCS are added to neutralize the action of the trypsin.

The suspension of cells is centrifuged at 1500 rev/min for 5 minutes, before taking up the pellet in 5 ml of PBS.

After a further centrifuging at 1500 rev/min for 5 minutes, 1 ml of a mixture of propidium iodide at 10 μg/ml and RNase at 1 mg/ml in solution in PBS is added. The preparation is incubated at ambient temperature for 30 minutes with the exclusion of light.

The contents of the preceding tubes are transferred into tubes suitable for FCM (Beckman-Coulter).

The samples are held at 4° C. until analysis.

Analysis

Analysis is carried out on a Beckman-Coulter FC500 cytometer with MXP software.

Autofluorescence controls (unlabeled cells) are used in order to avoid false positives and negatives.

The excitation of the nucleic acids/fluorochrome complex by the laser emitting at 488 nm produces an emission of the cell label in the red region (600-650 nm), the intensity of which is proportional to the amount of labeled DNA: a monoparametric linear histogram is constructed which makes it possible to measure the red fluorescence as a function of the number of cells.

The intensity of fluorescence is a function of the amount of DNA: the propidium iodide is fluorescent and binds to the DNA. The more DNA in the nucleus of each cell, the more intense the fluorescence signal. The cells containing 2n are in the phase of the cycle which precedes the synthesis of the DNA (G1) and the cells with the content 4n (after separation of the chromosomes) are in the phase which follows the synthesis (G2). The more the DNA content of between 2n and 4n is found, the more the cells are in the course of replicating (S phase).

Cursors separate the phases of the cell cycle into three groups: G0/G1, S and G2+M.

The cytometer analyzes the amount of fluorescence, which is proportional to the amount of labeled DNA, and converts it to percentages; the data of the analysis are expressed as percentage of cells occurring in each abovementioned phase for a total population of skin cells which represents 100%.

Results

The effects on aged fibroblasts of the treatment with the Vanda coerulea extract are studied.

The results obtained for the analysis by FCM of the treated or untreated NHFs are brought together in table 1 below (% s expressed with respect to the total number of cells):

TABLE 1

| | “Young” fibroblasts (F3P) | | “Aged” fibroblasts (F22P) | |
|---|---|---|---|---|
| | | Vanda | | Vanda |
| | Controls | coerulea | Controls | coerulea |
| G0/G1 phases | 84.1 | 84 | 91.1 | 83.91 |
| S phase | 4.6 | 4.8 | 1.2 | 4.7 |
| G2/M phases | 10.9 | 9.4 | 7.7 | 10.9 |

The results demonstrate a modification to the distribution of the cells according to the different phases of the cell cycle as a function of the number of passages. Specifically, it is observed, for the “aged” cells referred to as F22P cells, that their number in the G0/G1 phase of the cell cycle is greater than for the “young” cells referred to as F3P cells.

In the same way, the FCM results demonstrate a relatively smaller number of cells in the phase of replication of the DNA (S phase) and in mitosis (G2+M phase). This increase in the number of quiescent cells in these G0/G1 phases marks a general decrease in the cell activity. These cells are said to be pre-senescent.

Treatment of the young fibroblasts (F3P) with the Vanda coerulea extract does not modify the relative proportions of cells according to their involvement in the cell cycle, in comparison with the control (untreated F3P fibroblasts).

On the other hand, treatment of the aged fibroblasts (F22P) with the Vanda coerulea extract of example 1 makes it possible to restore a percentage of cells in the S phase of active replication of the genetic material equivalent to that of the untreated young fibroblasts (F3P).

The treatment makes it possible to “reverse” the phenomenon of pre-senescence observed for the untreated cells.

The treatment with the Vanda coerulea extract brings about a decrease in the percentage of senescent cells, blocked in the G0/G1 phase, and gives relatively more cells in the active phase of replication of the genetic information (S phase). The biological activity potential of the fibroblasts is thus increased. These surprising properties make possible use of the extract of the orchid Vanda coerulea as particularly effective cosmetic active agent for combating skin aging, in particular in the dermis, by increasing the number of active cells with respect to the number of senescent cells. This active principle is thus entirely innovative for combating well upstream from the cell aging process, in particular for restoring biological functions and mechanisms of “aged” fibroblasts.

### Example 3

### Anti-Aging Cream

The extract of Vanda coerulea stems used as active agent in this cosmetic composition is obtained by repeating the process of example 1 (extract No. 1). The dry extract is dissolved at 1% w/w in a glycerol/water 60/40 v/v mixture.

This extract solution is used as active agent in the preparation of the cosmetic composition below (% expressed as w/w):

Phase A

| | |
|---|---|
| 1% Solution of Vanda coerulea extract | 0.3 |
| Phenoxyethanol | 0.5 |
| Xanthan gum | 0.2 |
| Acrylates/C20-30 alkyl acrylate crosspolymers | 0.2 |
| Tetrasodium EDTA | 0.1 |
| Water | qs |

Phase B

| | |
|---|---|
| Hydrogenated polyisobutene | 4 |
| Squalane | 3 |
| Caprylic/capric triglyceride | 3 |
| Pentylene glycol | 3 |
| Glyceryl stearate | 3 |
| PEG-100 stearate | 2.5 |
| Beeswax | 1.5 |
| Dicaprylyl carbonate | 1.5 |
| Cetyl alcohol | 1 |
| Stearyl alcohol | 1 |
| Dimethicone | 1 |

Phase C

| | |
|---|---|
| Sodium hydroxide | 0.04 |
| Water | q.s. 100 |

The excipients of phase A are dispersed in the water and then heating is carried out to 80° C. before dissolving all the other compounds, including the aqueous/glycolic solution of Vanda coerulea extract.

The compounds of phase B are heated to 85° C. in order to form a homogeneous phase.

Phase A is emulsified in phase B using an Ystral mixer.

The oil-in-water emulsion thus obtained is finally neutralized using a 0.04% w/w aqueous sodium hydroxide solution and then cooled.

The composition obtained is an anti-aging cream intended to be applied to the face or a part of the face.

### Example 4

### Lightening Anti-Aging Serum

| | |
|---|---|
| Vanda coerulea extract according to example 1 | 0.2 |
| Madecassoside | 0.01 |
| Licorice extract | 0.05 |
| Oats polysaccharides | 3 |
| UV screening agents | 5 |
| Excipient | q.s. for 100 |

The preparation is applied in the morning to skin from which makeup has been removed.

1. A method of cosmetic care for delaying the appearance of signs of skin aging or to slow down the effects thereof, wherein said method comprising the application, to at least a part of the skin of the face or body, of an effective amount of a cosmetic composition comprising an extract of the orchid Vanda coerulea.

2. The cosmetic care method as claimed in claim 1, wherein said cosmetic composition is applied to an area of skin of the body or face exhibiting visible signs of aging, the presence of wrinkles or fine lines, a loss of skin elasticity, a reduction in the thickness of the skin, an increase in skin dryness or roughness, a loss of skin suppleness or radiance.

3. The method of claim 1, wherein said extract is an extract from a part selected from the group consisting of: stems, roots, leaves of said orchid Vanda coerulea.

4. The method of claim 1, wherein said extract is a polar solvent extract of said orchid Vanda coerulea.

5. The method of claim 4, wherein said polar solvent is selected from the group consisting of: water, C₁-C₄ alcohols, ethanol, C₂-C₆ glycols, glycerol, butylene glycol, propylene glycol, and any mixtures thereof.

6. The method of claim 5, wherein said solvent is an aqueous/alcoholic mixture.

7. The method of claim 4, wherein said polar solvent is in subcritical state.

8. The method of claim 1, wherein said extract has been subjected to at least one stage of discoloration or purification.

9. The method of claim 1, wherein said composition comprises from 0.001% to 5% by weight of dry extract.

10. The method of claim 1, wherein said composition additionally comprises at least one extract of another orchid.

11. The method of claim 1, wherein said composition additionally comprises at least one extract of another species of orchid belonging to the Vanda genus, or an extract of an orchid belonging to the genus Brassocattleya, or an extract of an orchid belonging to the genus Phalaenopsis.

12. The method of claim 1, wherein said composition additionally comprises at least one cosmetically acceptable excipient selected from the group consisting of: pigments, dyes, polymers, surface-active agents, rheology agents, fragrances, electrolytes, pH adjusters, antioxidants, preservatives and their mixtures.

13. The method of claim 1, wherein said cosmetic composition is a serum, a lotion, a cream, a hydrogel, a mask, a stick, or a patch.

14. A method of cosmetic care for maintaining the firmness of the skin and tissues or to restore a more radiant complexion to the skin, said method.

15. The method as claimed in claim 14, wherein said extract is an extract from a part selected from the group consisting of stems, roots, leaves of said orchid Vanda coerulea.

16. The method of claim 14, wherein said composition further comprises at least one extract of another orchid.

17. The method of claim 14, wherein said composition further comprises at least one extract of another species of orchid belonging to the Vanda genus, or an extract of an orchid belonging to the genus Brassocattleya, or an extract of an orchid belonging to the genus Phalaenopsis.

18. A method of cosmetic care for combating a pigmentation disorder related to skin aging, or visible signs of a disorder of pigmentation related to skin aging.

19. The method of claim 18, wherein said pigmentation disorder is a senile lentigo.

20. The method as claimed in claim 18, wherein said extract is an extract from a part selected from the group consisting of: stems, roots, leaves of said orchid Vanda coerulea.

21. The method of claim 18, wherein said composition further comprises at least one extract of another orchid.

22. The method of claim 18, wherein said composition further comprises at least one extract of another species of orchid belonging to the Vanda genus, or an extract of an orchid belonging to the genus Brassocattleya, or an extract of an orchid belonging to the genus Phalaenopsis.

23. A method of cosmetic care for regulating the cycle of the skin cells to increase the percentage of skin cells in the phase of replication of the genetic information and to reduce, at the same time, the percentage of senescent cells among the total population of skin cells, wherein said method comprises the application, to at least a part of the skin of the face or body, of an effective amount of a cosmetic composition comprising an extract of the orchid Vanda coerulea.

24. The method as claimed in claim 23, wherein said extract is an extract from a part selected from the group consisting of: stems, roots, leaves of said orchid Vanda coerulea.

25. The method of claim 23, wherein said composition further comprises at least one extract of another orchid.
